(19) United States
(12) Patent Application Publication
(10) Pub. No.: US 2009/0018416 A1
(43) Pub. Date: Jan. 15, 2009

(54) Analyte Concentration Measurement Device

(76) Inventors: Stephen D. Walker, Boulder, CO (US); Devon J. Dallmann, Boulder, CO (US); Charles W. Henry, Boulder, CO (US); Peter E. Nelson, Boulder, CO (US); R. Dale Zellers, Boulder, CO (US); John E. Repine, Boulder, CO (US)

Correspondence Address: KOESTNER BERTANI LLP, 2192 Martin St., Suite 150, Irvine, CA, 92612, US

(21) Appl. No.: 11/777,940

(22) Filed: Jul. 13, 2007

(51) Int. Cl. A61B 5/00

(52) U.S. Cl. 600/316

(57) ABSTRACT

A method for measuring concentration of an analyte in body fluid comprises acquiring a body fluid sample, emitting light into the body fluid sample, and detecting emitted light intensity on a plurality of optical paths through the body fluid sample. A plurality of optical filters are arranged in respective optical paths of the optical path plurality comprising at least a first optical filter with light absorption by an analyte and water and a second optical filter with light absorption to water alone. Light intensity passed through the first optical filter and passed through the second optical filter is measured and analyte concentration is determined based on a ratio of intensities detected at a detector in an optical path intersected by the first optical filter and detected at a detector in an optical path intersected by the second optical filter.

### BACKGROUND

The diabetic population is large and increasing. In 2005, 20.8 million Americans had diabetes, with over 1.5 million new cases diagnosed in the same year (American Diabetes Association (ADA) home page, www.diabetes.org). The diabetic population is growing by 7% annually, and shows little sign of abating (ADA home page, www.diabetes.org). Another 54 million Americans are pre-diabetic, meaning that they are already experiencing impaired glucose metabolism and up to 8% will become diabetic each year (Grady, D., Finding Whether Diabetes Lurks, New York Times, May 1, 2007).

Diabetic patients develop more medical complications and make up a disproportionate share of hospitalized patients. Diabetic or pre-diabetic patients comprise approximately 38% of all hospital admissions (Umpierrez G E, Isaacs S D, et al., Hyperglycemia: an independent marker of in-hospital mortality in patients with undiagnosed diabetes, Journal of Clinical Endocrinological Metabolism 2002; 87:978-982). Within hospital Intensive Care Units (ICUs) the percent of patients with impaired glucose metabolism is believed to be 56% (Davidson, Glucommander). Moreover, abnormal glucose metabolism also develops in seriously-ill non-diabetic individuals making the need for glucose assessment virtually universal.

Hospital care of patients with impaired glucose metabolism is shaped by three forces: (1) the vast number of diabetic patients; (2) the dramatic improvement in patient outcomes demonstrated by intensive insulin management; and (3) the very high cost of acquiring the frequent glucose measurements necessary to implement an intensive insulin therapy protocol.

Since the development of programs for intensive insulin management, improvement in the all-important measure of patient outcomes is well-documented. In 2001, Grete Van den Berghe, MD, published a seminal study that demonstrated the significant medical benefits derived by keeping an ICU patient's blood glucose levels between 80 and 110 mg/dl through highly managed insulin therapy (Van den Berghe G, et al., Intensive Insulin Therapy in Critically Ill Patients, New England Journal of Medicine (NEJM), Vol. 345, No. 19, Nov. 8, 2001). This study demonstrated very significant improvements in patient mortality, morbidity and length of hospitalization by aggressively using insulin to maintain low blood glucose levels and to decrease inflammation. Dr. Van den Berghe's initial findings have now been corroborated by many other studies in settings ranging from surgical ICUs (Furnary, A P, Zurr K J, et al, Continuous intravenous insulin infusion reduces the incidence of deep sternal wound infection in diabetic patients after cardiac surgical procedures. Annals of Thoracic Surgery 67:352-362, 1999) to general hospital wards (Newton, C A, Young, S, Financial implications of glycemic control, Endocrine Practice, Vol. 12, July/August 2006, p. 43-48) to organ transplantations. So why doesn't every hospital use an intensive insulin management protocol? The answer is cost.

The current finger-stick approach for measuring glucose in ICU patients is too expensive and cumbersome. Intensive blood glucose monitoring necessitates dedicating one hospital technician per every twelve ICU beds to collect blood glucose samples from finger sticks. Even with the aggressive approach of intensive monitoring, a new glucose value is generated only once every hour per patient and that value provides only a single data point of information from which to adjust insulin delivery rates. No method exists for real-time assessment of the glucose level's direction or rate of change. In seriously ill individuals, glucose and insulin levels and other factors which affect these levels are changing very rapidly. Thus, a need exists for more frequent measurements and the valuable trend data that more measurements provide. Despite the savings and the improved outcomes, many medical and surgical ICU's have not been able to embrace the intensive insulin therapy approach because tight glycemic control is difficult to accomplish in terms of staffing, training, implementing and managing. In particular, ICU patients must be guarded carefully against the development of low blood sugars (hypoglycemia). However, this concern needs to be balanced against the desire to give as much insulin and to reduce blood sugars are much as possible. The reason that lower blood glucose levels and administering insulin is life-saving is unknown but may relate to an ability to reduce inflammation which is a common and contributing factor in the illness of these patients. Although no proof drives the concept, avoiding large swings in blood glucose levels is believed to be beneficial and can be best accomplished if more frequent glucose readings are made and insulin administration can be titered more specifically and frequently.

Assuming that the average cost for each hourly glucose reading is $10 and that the average length of stay in the ICU is 3 days (72 hours), then $720 is spent per patient visit to collect hourly glucose values.

### SUMMARY

An embodiment of a method for measuring concentration of an analyte in body fluid comprises acquiring a body fluid sample, emitting light into the body fluid sample, and detecting emitted light intensity on a plurality of optical paths through the body fluid sample. A plurality of optical filters are arranged in respective optical paths of the optical path plurality comprising at least a first optical filter with light absorption by an analyte and water and a second optical filter with light absorption to water alone. Light intensity passed through the first optical filter and passed through the second optical filter is measured and analyte concentration is determined based on a ratio of intensities detected at a detector in an optical path intersected by the first optical filter and detected at a detector in an optical path intersected by the second optical filter.

### BRIEF DESCRIPTION OF THE DRAWINGS

Embodiments of the invention relating to both structure and method of operation may best be understood by referring to the following description and accompanying drawings:

FIG. 1 is a schematic block and pictorial diagram depicting an embodiment of an analyte concentration measurement apparatus;

FIGS. 2A through 2B are flow charts illustrating one or more embodiments or aspects of a method for measuring concentration of an analyte in body fluid;

FIG. 3 is a schematic block and pictorial diagram showing another embodiment of an analyte concentration measurement apparatus;

FIGS. 4A through 4B are flow charts illustrating one or more embodiments or aspects of another method for measuring concentration of an analyte in body fluid; and

FIG. 5 is a schematic block diagram showing an embodiment of a system that can be used for the illustrative analyte measurement devices and measurement methods.

### DETAILED DESCRIPTION

An improved method for measuring blood glucose levels is of paramount importance today for life-saving effects in severely-ill, hospitalized patients. The new technology depicted herein has the potential to improve patient diagnosis and care, while also reducing the medical expenses of the many diabetic and non-diabetic ICU patients in hospitals worldwide.

Example hospital sectors in which the illustrative analyte concentration measurement device and methods can make an immediate impact include intensive care units (ICUs), surgical, and general hospital applications.

In the intensive care unit (ICU) estimates are that by 2008, 70% of the 53,805 ICU beds in the US will use an intensive insulin management protocol.

In surgical sectors approximately 10% of the 31 million surgical procedures performed annually in the US are potential users of the analyte concentration measurement device. Anesthesia procedures of two hours or longer create an acute need for critical information on glucose excursions.

In a general hospital sector approximately 38% of a hospital's patient population has diabetes, is pre-diabetic, or has nutritional monitoring requirements. Assuming that only 15% of the general hospital patient population is penetrated, the general hospital sector is still 1½ times larger than that of the ICU and surgical opportunities combined.

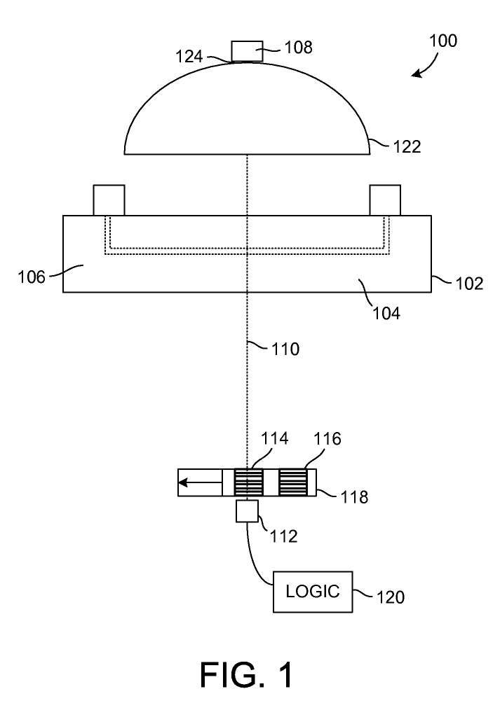

Referring to FIG. 1, a schematic block and pictorial diagram depicts an embodiment of an analyte concentration measurement apparatus 100 comprising a housing 102 enclosing a sample chamber 104 configured for holding a body fluid sample 106, an emitter 108 that emits light along an optical path 110 into the sample chamber 104, and a detector 112 positioned along the optical path 110 across the sample chamber 104 from the emitter 108 that detects emitted light intensity. The measurement apparatus 100 further comprises a first optical filter 114 with light absorption by an analyte and water, and a second optical filter 116 with light absorption to water alone. A switch 118 alternately interposes the first optical filter 114 and the second optical filter 116 into the optical path 110. A logic 120 determines the analyte concentration based on a ratio of intensities detected with the first optical filter 114 and the second optical filter 116 interposed into the optical path 110.

In a particular application, the apparatus 100 can comprise a glucose concentration measurement apparatus with the first optical filter 114 comprising a filter λ1 with light absorption by glucose and water, and the second optical filter 116 comprising a filter λ2 with light absorption by water alone. The logic 120 determines glucose concentration in the body fluid sample according to equation (1) as follows:

$${C}_{G} = \frac{\frac{{ɛ}_{{w}_{λ 1}}}{{ɛ}_{{w}_{λ 2}}} ln ( \frac{{I}_{{1}_{λ 2}}}{{I}_{{0}_{λ 2}}} ) - ln ( \frac{{I}_{{1}_{λ 1}}}{{I}_{{0}_{λ 1}}} )}{L {ɛ}_{{G}_{λ 1}}} , \qquad ( 1 )$$

where C_{G} is glucose molar fraction, L is path length through the body fluid sample, ε_{Gλ1} is glucose absorption coefficient at wavelength λ1, ε_{Wλ1} is water absorption coefficient at wavelength λ1, and ε_{Wλ2} is water absorption coefficient at wavelength λ2. I_{1λ1} is measured light intensity of wavelength λ1 through the body fluid sample, I_{0λ1} is light intensity of wavelength λ1 in absence of a sample in the sample chamber, I_{1λ2} is light intensity of wavelength λ2 through the body fluid sample in the sample chamber, and I_{0λ2} is light intensity of wavelength λ2 in absence of a sample 106 in the sample chamber 104.

In a first example application, the first optical filter 114 can be implemented as a filter with a light absorption wavelength λ1 of approximately 9.7 micrometers and the second optical filter 116 can be constructed as a filter with a light absorption wavelength λ2 of approximately 8.4 micrometers.

In a second example application, the first optical filter 114 can be implemented as a filter with a light absorption wavelength λ1 of approximately 9.0 micrometers and the second optical filter 116 can be constructed as a filter with a light absorption wavelength λ2 of approximately 8.4 micrometers.

In a particular arrangement, the first 114 and second 116 optical filters can be implemented as narrowband filters with a center wavelength variability of ±2%, a half power bandwidth of 0.12 micrometers, and peak transmission of 85%.

In various applications the housing 102 can enclose a sample chamber 104 configured for holding a body fluid sample such as plasma, serum, saliva, cerebrospinal fluid, tears, urine, extracellular fluids, or other fluid from a body that does not contain red blood cells or hemoglobin.

An implementation of a suitable housing 102 enclosing the sample chamber 104 can be formed of a material that is nonabsorbent to 8-10 micrometer light and is sufficiently rigid to maintain 0-50 micrometer spacing, and remains solid when contacted by body fluid. In a specific example, the housing 102 can be formed of high density polyethylene (HDPE) that has a transmission of approximately 53% at approximately 8.4 and 9.0 micrometers, and approximately 64% at approximately 9.7 micrometers.

In some embodiments, the measurement apparatus 100 can further comprise the emitter 108 in a configuration for radiating broadband infrared light and a parabolic reflector 122 which is separated by an air gap 124 from the emitter 108 and collimates the radiated broadband infrared light. The housing 102, emitter 108, and detector 112 can be arranged so that the optical path length is in a range of approximately 10-50 micrometers.

The switch 118 can be implemented as a sliding filter holder 126 whereby light passes through the selected filters 114, 116 held by the sliding filter holder 126 over the detector 112.

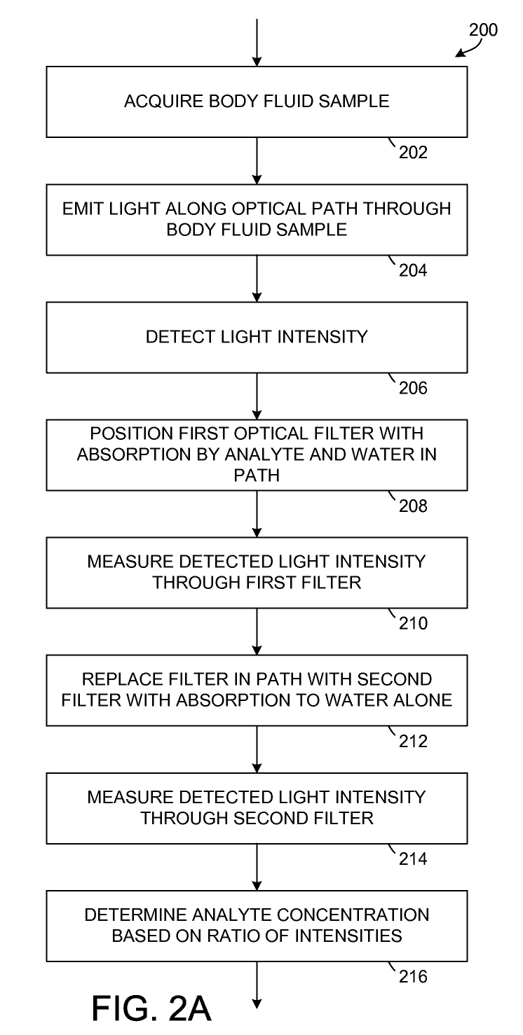

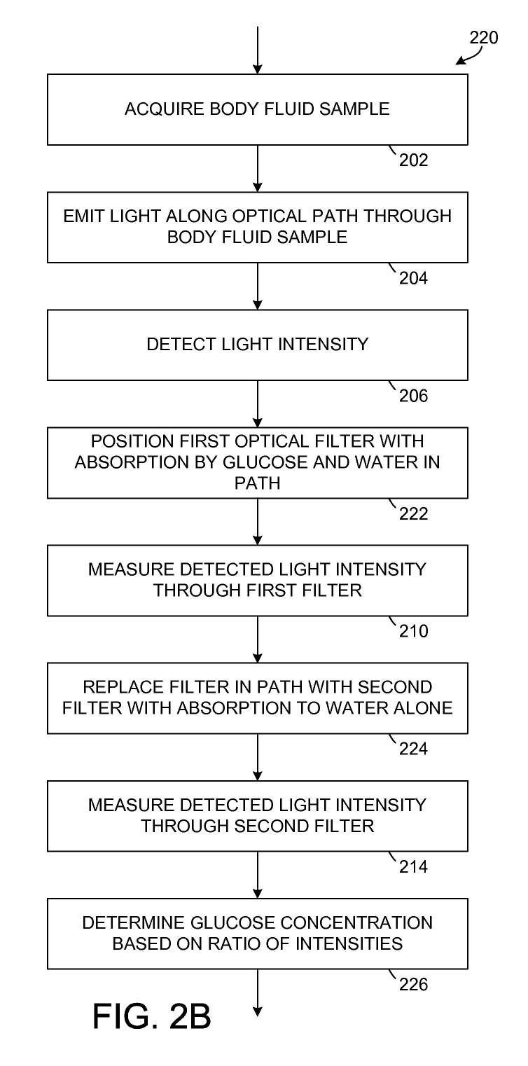

Referring to FIGS. 2A through 2B, flow charts illustrate one or more embodiments or aspects of a method for measuring 200 concentration of an analyte in body fluid. As shown in FIG. 2A, the analyte concentration measurement method 200 comprises acquiring 202 a body fluid sample, emitting 204 light along an optical path into the body fluid sample, and detecting 206 emitted light intensity on the optical path through the body fluid sample. A first optical filter with light absorption by an analyte and water can be positioned 208 in the optical path and the detected light intensity passed through the first optical filter is measured 210. The first optical filter can be replaced 212 with a second optical filter with light absorption to water alone and the detected light intensity passed through the second optical filter is measured 214. Analyte concentration is determined 216 based on a ratio of intensities detected with the first optical filter and the second optical filter interposed into the optical path.

The acquired body fluid sample can be, for example, plasma, serum, saliva, cerebrospinal fluid, tears, urine, extracellular fluids, or other fluid from a body that does not contain red blood cells or hemoglobin.

Referring to FIG. 2B, in a particular application glucose concentration can be measured 220 in body fluid by positioning 222 the first optical filter comprising a filter λ1 with light absorption by glucose and water, and replacing 224 the first optical filter with the second optical filter comprising a filter λ2 with light absorption by water alone. Glucose concentration in the body fluid sample is determined 226 according to equation (1).

In various applications, the first optical filter can be implemented as a filter with a light absorption wavelength λ1 of approximately 9.7 or 9.0 micrometers, for example, and the second optical filter with a light absorption wavelength λ2 of approximately 8.4 micrometers.

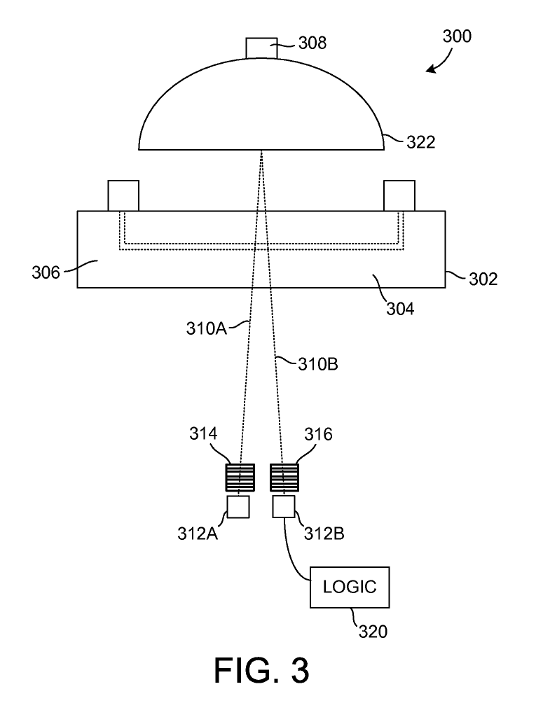

Referring to FIG. 3, a schematic block and pictorial diagram depicts an embodiment of an analyte concentration measurement apparatus 300 comprising a housing 302 enclosing a sample chamber 304 configured for holding a body fluid sample 306, an emitter 308 that emits light into the sample chamber 304, and a plurality of detectors 312 positioned along optical paths 310 across the sample chamber 304 from the emitter 308 that detect emitted light intensity. The measurement apparatus 300 further comprises a plurality of optical filters 314, 316 aligned in respective optical paths 310 of the detector plurality 312 including at least a first optical filter 314 with light absorption by an analyte and water, and a second optical filter 316 with light absorption to water alone. A logic 320 determines the analyte concentration based on a ratio of intensities detected at a first detector 312A in an optical path 310A intersected by the first optical filter 314 and detected at a second detector 312B in an optical path 310B intersected by the second optical filter 316.

In an illustrative embodiment, the apparatus 300 can be a glucose concentration measurement apparatus with the first optical filter 314 comprising a filter λ1 with light absorption by glucose and water and the second optical filter 314 comprising a filter λ2 with light absorption by water alone. The logic 320 determines glucose concentration in the body fluid sample according to equation (1).

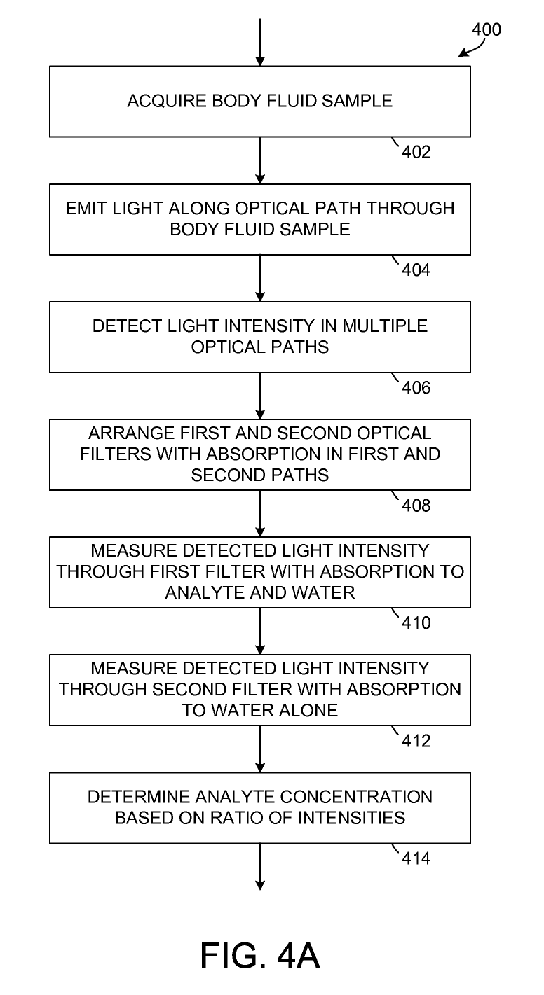

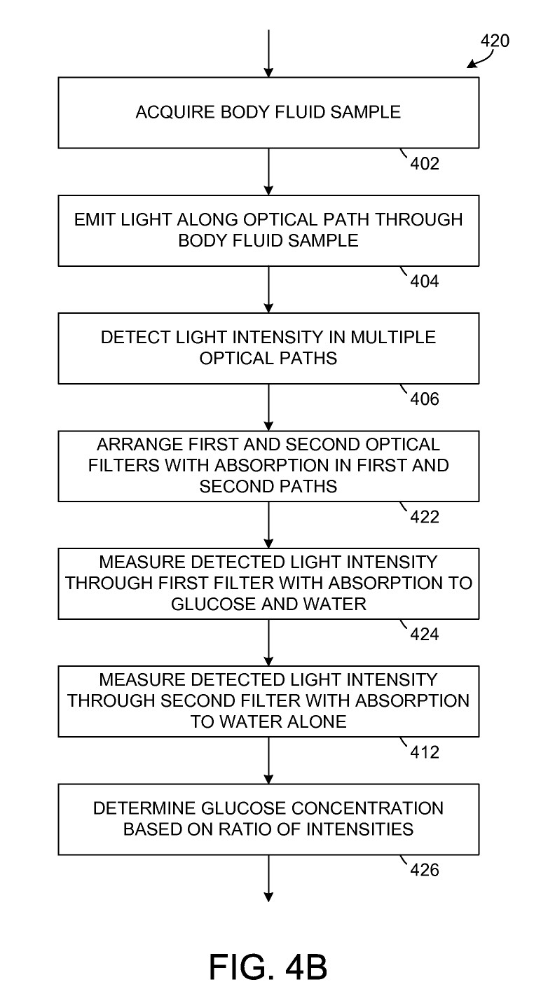

Referring to FIGS. 4A through 4B, flow charts illustrate one or more embodiments or aspects of a method for measuring 400 concentration of an analyte in body fluid. As shown in FIG. 4A, the analyte concentration measurement method 400 comprises acquiring 402 a body fluid sample, emitting 404 light into the body fluid sample, and detecting 406 emitted light intensity on multiple optical paths through the body fluid sample. Multiple optical filters are arranged 408 in respective optical paths of the optical paths including at least a first optical filter with light absorption by an analyte and water and a second optical filter with light absorption to water alone. The detected light intensity which is passed through the first optical filter is measured 410 and the detected light intensity which is passed through the second optical filter is measured 412. Analyte concentration is determined 414 based on a ratio of intensities detected at a detector in an optical path intersected by the first optical filter and detected at a detector in an optical path intersected by the second optical filter.

Referring to FIG. 4B, in a particular application glucose concentration can be measured 420 in body fluid by arranging 422 the optical filters including the first optical filter λ1 with light absorption by glucose and water, and the second optical filter λ2 with light absorption by water alone. Light intensity through the first filter with absorption to glucose and water is measured 424 and glucose concentration in the body fluid sample is determined 426 according to equation (1).

The concentration of glucose in a body fluid sample can be expressed by Beer's Law as shown in equation (2):

$${C}_{G} L {ɛ}_{λ} = - ln ( \frac{{I}_{1}}{{I}_{0}} ) , \qquad ( 2 )$$

where C_{G} is the glucose molar fraction, L is the path length, ε_{λ} is the glucose absorption coefficient at wavelength λ with units of cm⁻¹, I₀ is the light intensity of wavelength λ at the detector for no sample and I₁ is the light intensity of wavelength at the detector for a sample. However, usage of equation (2) is not practical for diagnostic purposes because other analytes present in body fluid, such as water, albumin, lipids and urea, also absorb infrared light. A wavelength that avoids water absorption cannot be selected due to water's high concentration in body fluid and a strong absorbance throughout the infrared region. Equation (3) describes Beer's Law for wavelength λ1 where only glucose and water have absorption:

$${C}_{G} L {ɛ}_{{g}_{λ 1}} + {C}_{w} L {ɛ}_{{w}_{λ 1}} = - ln ( \frac{{I}_{{1}_{λ 1}}}{{I}_{{0}_{λ1}}} ) , \qquad ( 3 )$$

where C_{G} is the glucose molar fraction, ε_{gλ1} is the glucose absorption coefficient at wavelength λ1, C_{w} is the water molar fraction, ε_{wλ1} is the water absorption coefficient at wavelength λ1, I_{0λ1} is the light intensity of wavelength λ1 at the detector for no sample and I_{1λ1} is the light intensity of wavelength λ1 at the detector for a sample. The water concentration can be determined as shown in equation (4) by measuring the light absorption at wavelength λ2 where only water has absorption:

$${C}_{w} L {ɛ}_{{w}_{λ 2}} = - ln ( \frac{{I}_{{1}_{λ2}}}{{I}_{{0}_{λ2}}} ) , \qquad ( 4 )$$

Glucose concentration is determined by passing light through the sample and through filter λ1. Light is also passed through the sample and through filter λ2. The wavelength for filter λ1 is selected to have absorption by both glucose and water while filter λ2 has only water absorption. Glucose concentration is found by substituting equation (4) into equation (3) to result in equation (5) as follows:

$${C}_{G} = \frac{\frac{{ɛ}_{{w}_{λ1}}}{{ɛ}_{{w}_{λ2}}} ln ( \frac{{I}_{{1}_{λ2}}}{{I}_{{0}_{λ2}}} ) - ln ( \frac{{I}_{{1}_{λ1}}}{{I}_{{0}_{λ1}}} )}{L {ɛ}_{{G}_{λ1}}} . \qquad ( 5 )$$

Referring again to FIG. 1, a schematic block diagram depicts an embodiment of an illustrative infrared glucose sensor 100. The infrared glucose sensor 100 measures glucose concentration in a body fluid sample 106 by determining the portion of light absorbed by glucose. An emitter 108 radiates broadband infrared light that is collimated with a parabolic shaped reflector 122. In an illustrative implementation, a 10 millimeter (mm) air gap is interposed between the reflector 122 and a sample chamber 104 to minimize thermal influences. The path length through the sample can be configured in a range between 10-50 micrometers (μm) to reduce or eliminate effects of strong water absorption. Light passes through one of two filters 114, 116 held by a sliding filter holder 118 over a detector 112. An alternate embodiment shown in FIG. 3 has two detectors, each with a filter.

Filters can be selected to enhance measurement of a desired analyte such as glucose. For example, the 9-10 micrometer range has glucose peaks at 9.0, 9.3 and 9.7 micrometers. However only the 9.0 and 9.7 micrometer values can be used for the λ1 filter because albumin masks the 9.3 μm absorbance. The 8.4 micrometer wavelength is selected for the λ2 filter because there is no absorption exists at the wavelength except for water. Filters can be narrowband to avoid interference from nearby analytes. Center wavelengths of the filters can have a tolerance range of ±2%, the half power bandwidth 0.12 micrometers, and the peak transmission 85%.

Similarly the sample chamber material can also be selected to improve measurement of the selected analyte. For example, the sample chamber material can be selected which does not absorb 8-10 micrometer light, is sufficiently rigid to hold 0-50 micrometer spacing, and does not dissolve when contacted by body fluid. Zinc selenide meets all the criteria but is expensive and difficult to clean. A sample chamber material that is low cost and disposable may be more desirable. One suitable such material is high density polyethylene (HDPE) that has a transmission of 53% at 8.4 and 9.0 micrometers, and 64% at 9.7 micrometers.

The sample also can be selected to facilitate measurement of the predetermined analyte. Plasma is a highly suitable sample due to abundance and an ability to be obtained at the patient bedside. Plasma is one of several body fluids that may be used as the sample. Other body fluids include serum, saliva, cerebrospinal fluids, tears, urine, extracellular fluids and any other fluids taken from the human body that do not contain red blood cells (RBCs) or hemoglobin. RBCs have a variable index of refraction and interfere with absorption measurements. Glucose level, oxygenation level, pH and temperature are some of the factors affecting RBC index of refraction. Hemoglobin absorbs light at 9.0 micrometers and interferes with glucose measurement at that wavelength.

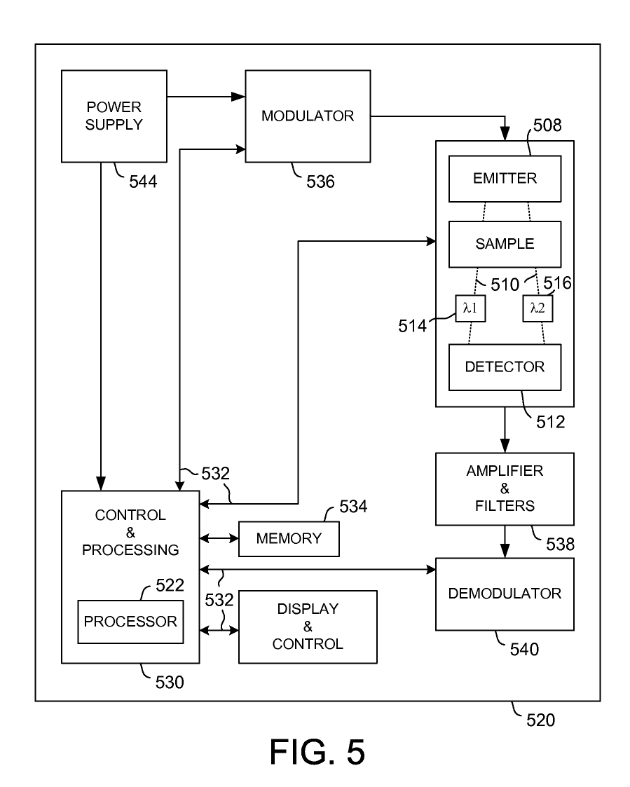

Referring to FIG. 5, a schematic block diagram illustrates an embodiment of a system 520 that can be used for the illustrative analyte measurement devices and measurement methods.

In an illustrative embodiment, a control and processing board 530 supports control/processing operations. A processor 522 controls the sensor 500 and supports hardware through an I²C serial bus 532. The processor 522 measures the light intensity I_{1λ2} for 10 seconds with filter λ1 514 in the optical path 510, moves filter λ2 516 into the optical path 510, measures the light intensity I_{1λ2} for 10 seconds with filter λ2 516, computes the average of both intensities and calculates the glucose concentration using equation (5). Glucose concentration is displayed and stored in combination with the 10 second intensities on a secure digital (SD) memory card 534.

An illustrative system 520 also includes a modulator 536. The modulator 536 uses a 2.0 megahertz (MHz) crystal oscillator-produced square wave signal and passes the signal through a series of counters to divide down to a 7-8 hertz (Hz) square wave. The reduced-frequency square wave is used to turn the emitter 508 on and off, allowing a periodic change in the light intensity on the detector 512.

The emitter 508 can be implemented as a broadband mid-infrared source that emits light over the 1-20 micrometer range. Intex M IRL 17-900-R is an emitter device that meets the criteria and has a parabolic reflector built into the device directly behind the emitter to collimate and focus the emitted light. The drive voltage for the emitter 508 can be supplied by a LT1129 programmable linear regulator.

A detector 512 converts changes in incident infrared energy into voltage. A suitable detector 512 is the InfraTech LIE-345 pyroelectric detector.

A signal from the detector 512 can be passed to an amplifier and filters 538 including a notch filter at 60 Hz to reduce 60 Hz noise induced by surrounding electrical sources. The notch filter has built in amplification under 60 Hz and a reduced gain above the 60 Hz notch. Amplification of the pre-notch and post-notch frequencies can be changed by changing resistor values. The output of the amplifier/filter 538 is fed into a demodulator 540.

The demodulator 540 receives the amplified and filtered detector output signal and converts the signal into a DC level by taking the difference between the on and off states of the square wave. The demodulator 540 suppresses voltage fluctuations outside the 7-8 Hz range. The demodulator 540 can have a programmable phase adjustment that allows the phase of the demodulator 540 to match the phase of the modulator 536. The output signal from the demodulator 540 is sampled using an analog-to-digital converter.

The illustrative system 520 further comprises a power supply 544 which can be a switching 85-264V RMS 47-63 Hz AC to 12V DC 7 watt medical grade power supply.

Terms “substantially”, “essentially”, or “approximately”, that may be used herein, relate to an industry-accepted tolerance to the corresponding term. Such an industry-accepted tolerance ranges from less than one percent to twenty percent and corresponds to, but is not limited to, functionality, values, process variations, sizes, operating speeds, and the like. The term “coupled”, as may be used herein, includes direct coupling and indirect coupling via another component, element, circuit, or module where, for indirect coupling, the intervening component, element, circuit, or module does not modify the information of a signal but may adjust its current level, voltage level, and/or power level. Inferred coupling, for example where one element is coupled to another element by inference, includes direct and indirect coupling between two elements in the same manner as “coupled”.

The illustrative block diagrams and flow charts depict process steps or blocks that may represent modules, segments, or portions of code that include one or more executable instructions for implementing specific logical functions or steps in the process. Although the particular examples illustrate specific process steps or acts, many alternative implementations are possible and commonly made by simple design choice. Acts and steps may be executed in different order from the specific description herein, based on considerations of function, purpose, conformance to standard, legacy structure, and the like.

While the present disclosure describes various embodiments, these embodiments are to be understood as illustrative and do not limit the claim scope. Many variations, modifications, additions and improvements of the described embodiments are possible. For example, those having ordinary skill in the art will readily implement the steps necessary to provide the structures and methods disclosed herein, and will understand that the process parameters, materials, and dimensions are given by way of example only. The parameters, materials, and dimensions can be varied to achieve the desired structure as well as modifications, which are within the scope of the claims. Variations and modifications of the embodiments disclosed herein may also be made while remaining within the scope of the following claims.

What is claimed is:

1. Analyte concentration measurement apparatus comprising:
a housing enclosing a sample chamber configured for holding a body fluid sample;
an emitter that emits light along an optical path into the sample chamber;
a detector positioned along the optical path across the sample chamber from the emitter that detects emitted light intensity;
a first optical filter with light absorption by an analyte and water;
a second optical filter with light absorption to water alone;
a switch that alternately interposes the first optical filter and the second optical filter into the optical path; and
a logic that determines the analyte concentration based on a ratio of intensities detected with the first optical filter and the second optical filter interposed into the optical path.

2. The apparatus according to claim 1 further comprising:
the apparatus comprising a glucose concentration measurement apparatus;
the first optical filter comprising a filter λ1 with light absorption by glucose and water;
the second optical filter comprising a filter λ2 with light absorption by water alone; and
the logic determines glucose concentration in the body fluid sample according to an equation as follows:
$${C}_{G} = \frac{\frac{{ɛ}_{{w}_{λ1}}}{{ɛ}_{{w}_{λ2}}} ln ( \frac{{I}_{{1}_{λ2}}}{{I}_{{0}_{λ2}}} ) - ln ( \frac{{I}_{{1}_{λ1}}}{{I}_{{0}_{λ1}}} )}{L {ɛ}_{{G}_{λ1}}} ,$$
where C_{G} is glucose molar fraction, L is path length through the body fluid sample, ε_{Gλ1} is glucose absorption coefficient at wavelength λ1, ε_{Wλ1} is water absorption coefficient at wavelength λ1, ε_{Wλ2} is water absorption coefficient at wavelength λ2, I_{1λ1} is measured light intensity of wavelength λ1 through the body fluid sample, I_{0λ1} is light intensity of wavelength λ1 in absence of a sample in the sample chamber, I_{1λ2} is light intensity of wavelength λ2 through the body fluid sample in the sample chamber, and I_{0λ2} is light intensity of wavelength λ2 in absence of a sample in the sample chamber.

3. The apparatus according to claim 2 further comprising:
the first optical filter comprising a filter with a light absorption wavelength λ1 of approximately 9.7 micrometers; and
the second optical filter comprising a filter with a light absorption wavelength λ2 of approximately 8.4 micrometers.

4. The apparatus according to claim 2 further comprising:
the first optical filter comprising a filter with a light absorption wavelength λ1 of approximately 9.0 micrometers; and
the second optical filter comprising a filter with a light absorption wavelength λ2 of approximately 8.4 micrometers.

5. The apparatus according to claim 1 further comprising:
the housing enclosing a sample chamber configured for holding a body fluid sample comprising plasma, serum, saliva, cerebrospinal fluid, tears, urine, extracellular fluids, or other fluid from a body that does not contain red blood cells or hemoglobin.

6. The apparatus according to claim 1 further comprising:
the emitter configured to radiate broadband infrared light;
a parabolic reflector separated by an air gap from the emitter that collimates the radiated broadband infrared light; and
the housing, emitter, and detector arranged whereby optical path length is in an approximate range of 10-50 micrometers.

7. The apparatus according to claim 1 further comprising:
the switch comprising a sliding filter holder whereby light passes through a selected filters held by the sliding filter holder over the detector.

8. The apparatus according to claim 1 further comprising:
the first and second optical filters comprising narrowband filters with a center wavelength variability of ±2%, a half power bandwidth of 0.12 micrometers, and peak transmission of 85%.

9. The apparatus according to claim 1 further comprising:
the housing enclosing the sample chamber is formed of a material that is nonabsorbent to 8-10 micrometer light and is sufficiently rigid to maintain 0-50 micrometer spacing, and remains solid when contacted by body fluid.

10. The apparatus according to claim 1 further comprising:
the housing enclosing the sample chamber is formed of high density polyethylene (HDPE) that has a transmission of approximately 53% at approximately 8.4 and 9.0 micrometers, and approximately 64% at approximately 9.7 micrometers.

11. A method for measuring concentration of an analyte in body fluid comprising:
acquiring a body fluid sample;
emitting light along an optical path into the body fluid sample;
detecting emitted light intensity on the optical path through the body fluid sample;
positioning a first optical filter with light absorption by an analyte and water in the optical path;
measuring the detected light intensity passed through the first optical filter;
replacing the first optical filter with a second optical filter with light absorption to water alone;
measuring the detected light intensity passed through the second optical filter;
determining analyte concentration based on a ratio of intensities detected with the first optical filter and the second optical filter interposed into the optical path.

12. The method according to claim 11 further comprising:
measuring glucose concentration in body fluid;
positioning the first optical filter comprising a filter λ1 with light absorption by glucose and water;
replacing the first optical filter with the second optical filter comprising a filter λ2 with light absorption by water alone; and
determining glucose concentration in the body fluid sample according to an equation as follows:
$${C}_{G} = \frac{\frac{{ɛ}_{{w}_{λ1}}}{{ɛ}_{{w}_{λ2}}} ln ( \frac{{I}_{{1}_{λ2}}}{{I}_{{0}_{λ2}}} ) - ln ( \frac{{I}_{{1}_{λ1}}}{{I}_{{0}_{λ1}}} )}{L {ɛ}_{{G}_{λ1}}} ,$$
where C_{G} is glucose molar fraction, L is path length through the body fluid sample, ε_{Gλ1} is glucose absorption coefficient at wavelength λ1, ε_{Wλ1} is water absorption coefficient at wavelength λ1, ε_{Wλ2} is water absorption coefficient at wavelength λ2, I_{1λ1} is measured light intensity of wavelength λ1 through the body fluid sample, I_{0λ1} is light intensity of wavelength λ1 in absence of a sample in the sample chamber, I_{1λ2} is light intensity of wavelength λ2 through the body fluid sample in the sample chamber, and I_{1λ2} is light intensity of wavelength λ2 in absence of a sample in the sample chamber.

13. The method according to claim 12 further wherein:
the first optical filter comprises a filter with a light absorption wavelength λ1 of approximately 9.7 micrometers; and
the second optical filter comprises a filter with a light absorption wavelength λ2 of approximately 8.4 micrometers.

14. The method according to claim 12 wherein:
the first optical filter comprises a filter with a light absorption wavelength λ1 of approximately 9.0 micrometers; and
the second optical filter comprises a filter with a light absorption wavelength λ2 of approximately 8.4 micrometers.

15. The method according to claim 11 further comprising:
acquiring the body fluid sample comprising plasma, serum, saliva, cerebrospinal fluid, tears, urine, extracellular fluids, or other fluid from a body that does not contain red blood cells or hemoglobin.

16. Analyte concentration measurement apparatus comprising:
a housing enclosing a sample chamber configured for holding a body fluid sample;
an emitter that emits light into the sample chamber;
a plurality of detectors positioned along optical paths across the sample chamber from the emitter that detect emitted light intensity;
a plurality of optical filters aligned in respective optical paths of the detector plurality comprising at least a first optical filter with light absorption by an analyte and water and a second optical filter with light absorption to water alone; and
a logic that determines the analyte concentration based on a ratio of intensities detected at a first detector in an optical path intersected by the first optical filter and detected at a second detector in an optical path intersected by the second optical filter.

17. The apparatus according to claim 16 further comprising:
the apparatus comprising a glucose concentration measurement apparatus;
the first optical filter comprising a filter λ1 with light absorption by glucose and water;
the second optical filter comprising a filter λ2 with light absorption by water alone; and
the logic determines glucose concentration in the body fluid sample according to an equation as follows:
$${C}_{G} = \frac{\frac{{ɛ}_{{w}_{λ1}}}{{ɛ}_{{w}_{λ2}}} ln ( \frac{{I}_{{1}_{λ2}}}{{I}_{{0}_{λ2}}} ) - ln ( \frac{{I}_{{1}_{λ1}}}{{I}_{{0}_{λ1}}} )}{L {ɛ}_{{G}_{λ1}}} ,$$
where C_{G} is glucose molar fraction, L is path length through the body fluid sample, ε_{Gλ1} is glucose absorption coefficient at wavelength λ1, ε_{Wλ1} is water absorption coefficient at wavelength λ1, ε_{Wλ2} is water absorption coefficient at wavelength λ2, I_{1λ1} is measured light intensity of wavelength λ1 through the body fluid sample, I_{0λ1} is light intensity of wavelength λ1 in absence of a sample in the sample chamber, I_{1λ2} is light intensity of wavelength λ2 through the body fluid sample in the sample chamber, and I_{0λ2} is light intensity of wavelength λ2 in absence of a sample in the sample chamber.

18. The apparatus according to claim 17 further comprising:
the first optical filter comprising a filter with a light absorption wavelength λ1 of approximately 9.7 micrometers; and
the second optical filter comprising a filter with a light absorption wavelength λ2 of approximately 8.4 micrometers.

19. The apparatus according to claim 17 further comprising:
the first optical filter comprising a filter with a light absorption wavelength λ1 of approximately 9.0 micrometers; and
the second optical filter comprising a filter with a light absorption wavelength λ2 of approximately 8.4 micrometers.

20. The apparatus according to claim 16 further comprising:
the housing enclosing a sample chamber configured for holding a body fluid sample comprising plasma, serum, saliva, cerebrospinal fluid, tears, urine, extracellular fluids, or other fluid from a body that does not contain red blood cells or hemoglobin.

21. The apparatus according to claim 16 further comprising:
the emitter configured to radiate broadband infrared light;
a parabolic reflector separated by an air gap from the emitter that collimates the radiated broadband infrared light; and
the housing, emitter, and detectors arranged whereby optical path lengths are in an approximate range of 10-50 micrometers.

22. The apparatus according to claim 16 further comprising:
the first and second optical filters comprising narrowband filters with a center wavelength variability of ±2%, a half power bandwidth of 0.12 micrometers, and peak transmission of 85%.

23. The apparatus according to claim 16 further comprising:
the housing enclosing the sample chamber is formed of a material that is nonabsorbent to 8-10 micrometer light and is sufficiently rigid to maintain 0-50 micrometer spacing, and remains solid when contacted by body fluid.

24. The apparatus according to claim 16 further comprising:
the housing enclosing the sample chamber is formed of high density polyethylene (HDPE) that has a transmission of approximately 53% at approximately 8.4 and 9.0 micrometers, and approximately 64% at approximately 9.7 micrometers.

25. A method for measuring concentration of an analyte in body fluid comprising:
acquiring a body fluid sample;
emitting light into the body fluid sample;
detecting emitted light intensity on a plurality of optical paths through the body fluid sample;
arranging a plurality of optical filters in respective optical paths of the optical path plurality comprising at least a first optical filter with light absorption by an analyte and water and a second optical filter with light absorption to water alone;
measuring the detected light intensity passed through the first optical filter;
measuring the detected light intensity passed through the second optical filter;
determining analyte concentration based on a ratio of intensities detected at a detector in an optical path intersected by the first optical filter and detected at a detector in an optical path intersected by the second optical filter.

26. The method according to claim 25 further comprising:
measuring glucose concentration in body fluid;
arranging the optical filters including the first optical filter comprising a filter λ1 with light absorption by glucose and water, and the second optical filter comprising a filter λ2 with light absorption by water alone; and
determining glucose concentration in the body fluid sample according to an equation as follows:
$${C}_{G} = \frac{\frac{{ɛ}_{{w}_{λ1}}}{{ɛ}_{{w}_{λ2}}} ln ( \frac{{I}_{{1}_{λ2}}}{{I}_{{0}_{λ2}}} ) - ln ( \frac{{I}_{{1}_{λ1}}}{{I}_{{0}_{λ1}}} )}{L {ɛ}_{{G}_{λ1}}} ,$$
where C_{G} is glucose molar fraction, L is path length through the body fluid sample, ε_{Gλ1} is glucose absorption coefficient at wavelength λ1, ε_{Wλ1} is water absorption coefficient at wavelength λ1, ε_{Wλ2} is water absorption coefficient at wavelength λ2, I_{1λ1} is measured light intensity of wavelength λ1 through the body fluid sample, I_{0λ1} is light intensity of wavelength λ1 in absence of a sample in the sample chamber, I_{1λ2} is light intensity of wavelength λ2 through the body fluid sample in the sample chamber, and I_{0λ2} is light intensity of wavelength λ2 in absence of a sample in the sample chamber.

27. The method according to claim 26 further wherein:
the first optical filter comprises a filter with a light absorption wavelength λ1 of approximately 9.7 micrometers; and
the second optical filter comprises a filter with a light absorption wavelength λ2 of approximately 8.4 micrometers.

28. The method according to claim 26 wherein:
the first optical filter comprises a filter with a light absorption wavelength λ1 of approximately 9.0 micrometers; and
the second optical filter comprises a filter with a light absorption wavelength λ2 of approximately 8.4 micrometers.

29. The method according to claim 25 further comprising:
acquiring the body fluid sample comprising plasma, serum, saliva, cerebrospinal fluid, tears, urine, extracellular fluids, or other fluid from a body that does not contain red blood cells or hemoglobin.
